(19) United States
(12) Patent Application Publication
(10) Pub. No.: US 2005/0014719 A1
(43) Pub. Date: Jan. 20, 2005

(54) Method of controlling viral outbreak

(76) Inventor: Adil A. Khan, Baltimore, MD (US)

Correspondence Address: Mr. Adil A. Khan, 2606 N. Calvert St., Baltimore, MD, 21218, US

(21) Appl. No.: 10/877,659

(22) Filed: Jun. 24, 2004

Related U.S. Application Data
(60) Provisional application No. 60/482,097, filed on Jun. 24, 2003.

(51) Int. Cl. A61K 31/724

(52) U.S. Cl. 514/58

(57) ABSTRACT

This invention provides a novel application for β-cyclodextrin, a cholesterol depletor, as an inhibitor of viral outbreak. Topical applications of β-cyclodextrin are recommended to inhibit or reduce the severity of viral outbreaks such as oral or genital herpes. This invention also provides a novel technique for the creation of immunogens. Viral entry and outbreak also occurs at specialized lipid raft domains and disruption of rafts with cholesterol depletors blocks viral entry and outbreak (budding). Lipid microdomains (lipid rafts) are mobile regions of the plasma membrane and exist in all mammalian cell membranes. They are produced by the preferential packing of cholesterol and sphingolipids into the plasma membrane and are identified by their low solubility in detergents and enrichment with gangliosides such as G_{M1}. The size and composition of rafts can be dynamically altered during transmembrane signaling. Depletion of membrane cholesterol disrupts lipid rafts and inhibits viral entry and outbreak. Virus entry into cells involves virus/lipid raft interaction wherein the virus unfolds to enter the cell via the lipid raft. I provide a technique for the creation of an immunogen with novel viral epitopes based on the virus/lipid raft interaction and viral unfolding. Viral unfolding only occurs in lipid rafts. This fact can be exploited to create novel immunogens based on viral interaction with lipid rafts. The virus/lipid raft co-culture technique will create novel immunogens which will be used to create novel neutralizing monoclonal and polyclonal antibodies to fight viral disease such as HIV infection.

This application claims the benefit of U.S. provisional application No. 60/482,097, filed Jun. 24rd, 2003, the entire content of which is hereby incorporated herein by reference.

### FIELD OF THE INVENTION

The present invention relates to a novel method of creating an immunogen and using it to produce antibodies against nonenveloped and enveloped viruses, bacterial pathogens, fungal pathogens, other microbial pathogens, and proteins.

The invention relates generally to agents and methods for preventing a viral outbreak and, more specifically, to compositions containing a β-cyclodextrin (β-CD) and methods of using such compositions to decrease the probability and/or reduce the severity of a viral outbreak.

The present invention also relates to a pharmaceutical composition, which includes 1) β-CD, which is in a sufficient amount to block viral passage through lipid rafts in the membrane of nerve cells.

The present invention further relates to a composition, comprising a solid substrate that contains an effective amount of β-CD useful for reducing viral release.

### BACKGROUND OF THE INVENTION

The plasma membrane of immune and non-immune cells is composed of detergent-insoluble domains called lipid rafts, which are membrane compartments enriched in cholesterol and sphingolipids. In some tissues these specialized domains are referred to as caveolae. The initiation and propagation of intracellular signaling events occurs in these specialized membrane regions. Lipid rafts also contain many lipid-modified signaling proteins, and restrict their diffusion. Some examples of proteins associated with lipid rafts are tyrosine kinases of the Src family, glycophosphatidylinositol (GPI)-linked proteins, as well as adaptor proteins.

The confinement of signaling molecules to membrane subdomains suggests that lipid rafts are platforms for the formation of multicomponent transduction complexes. When immune receptors binds to their ligand, they become associated with lipid rafts. Additional components of the receptor signaling pathways are subsequently recruited to the rafts and form macromolecular signaling complexes. The initial translocation of immune receptors into lipid rafts is an important step in regulating cell activation.

Numerous experiments have provided substantial evidence that the integrity of lipid rafts is crucial for the initiation and maintenance of intracellular signals. Depletion of cholesterol, a component of lipid rafts, has been shown to inhibit HIV infection and illustrates the importance of lipid rafts in viral infection. Virus fusion and entry involves sequential interactions between viral proteins and proteins of the cell surface. These fusion and entry interactions proceed via three-dimensional rearrangements of viral and cell-surface proteins, thus giving rise to novel, but transient antigenic features. The present invention exploits those unique antigenic features to create novel anti-viral antibodies.

Virus entry into host cells involves the specific interaction of virus with receptor molecules contained within. Virus assembly and increases in viral concentration also occur in lipid rafts. Thus, the interaction between virus particles and lipid rafts presents an environment in which novel viral epitopes may be exposed. The interaction of proteins and lipid rafts generates novel configurations of the proteins that may be exploited to produce novel antibodies against the protein.

The significance of detergent-insoluble, glycolipid-enriched membrane domains (“lipid rafts”) has been demonstrated, particularly in regard to activation and signaling in T lymphocytes. Lipid rafts can be viewed as floating rafts comprised of sphingolipids and cholesterol that sequester glycosylphosphatidylinositol (GPI)-linked proteins such as Thy-1 and CD59. CD45, a 200 kDa transmembrane phosphatase protein, is excluded from these domains. Human immunodeficiency virus type 1 (HIV-1) particles produced by infected T cell lines acquire the GPI-linked proteins Thy-1 and CD59, as well as the ganglioside GM1, which is known to partition preferentially into lipid rafts. In contrast, despite its high expression on the cell surface, CD45 is poorly incorporated into virus particles. Confocal fluorescence microscopy revealed that HIV-1 proteins colocalized with Thy-1, CD59, GM1, and a lipid raft-specific fluorescent lipid, DiIC16 (see below), in uropods of infected Jurkat cells. CD45 did not colocalize with HIV-1 proteins and was excluded from uropods. Dot immunoassay of Triton X-100-extracted membrane fractions revealed that HIV-1 p17 matrix protein and gp41 were present in the detergent-resistant fractions and that (³H)-myristic acid-labeled HIV Gag protein showed a nine-to-one enrichment in lipid rafts. As disclosed herein, the budding of HIV virions through lipid rafts is associated with the presence of host cell cholesterol, sphingolipids, and GPI-linked proteins within these domains in the viral envelope, indicating preferential sorting of HIV Gag to lipid rafts (see Example 1).

Glycolipid-enriched membrane (GEM) domains are organized areas on the cell surface enriched in cholesterol, sphingolipids, and GPI-linked proteins. These domains have been described as “rafts” that serve as moving platforms on the cell surface (Shaw and Dustin, Immunity. 6:361-369, 1997). The domains, now referred to as “lipid rafts,” exist in a more ordered state, conferring resistance to Triton X-100 detergent treatment at 4° C. (Schroeder et al., J. Biol. Chem. 273:1150-1157, 1998). Many proteins are associated with lipid rafts, including GPI-linked proteins, Src family kinases, protein kinase C, actin and actin-binding proteins, heterotrimeric and small G proteins, and caveolin (see, for example, Arni et al., Biochem. Biophys. Res. Commun. 225:8001-807, 1996; Cinek and Horejsi, J. Immunol. 149:2262-2270, 1992; Robbins et al., Mol. Cell. Biol. 15:3507-3515, 1995; and Sargiacomo et al., J. Cell. Biol. 122:789-807, 1993). Saturated acyl chains of the GPI anchor have been shown to be a determinant for the association of GPI-linked proteins with lipid rafts (Rodgers et al., Mol. Cell. Biol. 14:5384-5391, 1994; Schroeder et al., Proc. Natl. Acad. Sci., USA. 91:12130-12134, 1994). Lipid rafts exclude certain transmembrane molecules, specifically the membrane phosphatase CD45 (Arne et al., supra, 1996; Rodgers and Rose, J. Cell. Biol. 135:1515-1523, 1996). Exclusion of CD45 results in the accumulation of phosphorylated signaling molecules in lipid rafts, and T cell activation requires clustering of signaling molecules in these membrane domains (Lanzavecchia et al., Cell. 96:1-4, 1999).

The role of lipid rafts in viral infection can further be extended to viruses other than HIV. For example, selective budding occurs for a virus of the influenza family, fowl plague virus, from ordered lipid domains (Scheiffele et al., J. Biol. Chem. 274:2038-2044, 1999, which is incorporated herein by reference). The requirement for cholesterol and sphingolipids in target membranes for Semliki Forest virus fusion also has been established (Nieva et al., EMBO J. 13:2797-2804, 1994; Phalen and Kielian, J. Cell Biol. 112:615-623, 1991, each of which is incorporated herein by reference). The interactions of lipid rafts with accessory HIV-1 molecules such as Vif and Nef can have important roles in virus budding, since interactions of myristylated HIV and simian immunodeficiency virus Nef with Lck, which is present in lipid rafts, and its incorporation into virions have been established (see, for example, Collette et al., J. Biol. Chem. 271:6333-6341, 1996; Flahertyet al., AIDS Res. Hum. Retrovir. 14:163-170, 1998).

Example 1 describes the interaction of HIV virus with lipid raft resident molecules such as GM1. The results disclosed in Example 1 indicate that HIV-1 buds through lipid rafts. During the course of infection, the cell becomes activated and polarization occurs, capping normally dispersed lipid rafts along with GPI-linked proteins and associated intracellular signaling molecules, and membrane areas containing CD45 can be cleared out of the cap site. The newly translated viral Gag precursor protein associated with lipid rafts then can be directed to the capped pole, where assembly and budding occurs. Palmitylated gp41 (gp160) is also directed into lipid rafts, and the interaction of its cytoplasmic tail with Gag protein in lipid rafts can prevent its internalization, allowing for the incorporation of gp160 into virions only at the site of budding (see Egan et al., J. Virol. 70:6547-6556, 1996; Yu et al., J. Virol. 66:4966-4971, 1992). Individual targeting of Gag and Env to the same site at the membrane can be an important means for delivering these proteins to the site of budding, since Gag and Env are processed and transported in different pathways within the cell. The host membrane then can become the new viral coat, resulting in the incorporation of cholesterol, sphingolipids, Thy-1, and CD59 and in the exclusion of CD45. HIV-1 also acquires functional adhesion molecules from host cells (Orentas and Hildreth, supra, 1993). These host-acquired proteins can significantly affect the biology of HIV-1 (see, for example, Fortin et al., J. Virol. 71:3588-3596, 1997).

### BRIEF SUMMARY OF INVENTION

The present invention relates to a novel method of designing an immunogen and producing antibodies to nonenveloped and enveloped viruses, and proteins. Specifically, it uses the co-culture of purified lipid rafts and viral particles as an immunogen.

The present invention also relates to a novel therapeutic method of preventing viral outbreaks (budding), using β-cyclodextrins.

Viral entry into cells involves unfolding of the virus and penetration into the cell at specific lipid raft sites. Antibodies created against the lipid raft/virus co-culture exploit viral unfolding to reveal novel epitopes in the virus that may be exploited as immunogens to create novel neutralizing antibodies. Purified preparations of lipid rafts are easily prepared from primary lymphoctes or transformed lymphocytes such as the Jurkat cell line. Purified isolates of HIV obtained from infected cell supernatants can be co-cultured with purified fractions of lipid rafts. These co-cultures can be used intact as immunogens or partially proteolysed to create viral/raft fragments. Additionally, the viral/raft co-cultures can remain co-cultured intact or fixed while co-cultured in mild fixative such as paraformaldehyde or glutaraldehyde. The steps involve mixing purified raft fractions with isolated virus. This admixture of raft/virus serves as the novel immunogen. Antibodies created against the lipid raft/virus co-culture will recognize antigenic determinants of the virus unique to the lipid raft/virus interaction. The method employs lipid raft/virus or lipid raft/protein suspensions as immunogens to develop polyclonal or monoclonal antibodies. Alternatively, lipid raft fractions may be made from infected cells such as lymphocytes or an immune cell line such as Jurkat. Lipid rafts produced in this fashion would already contain interactive virus and would be ready for use as a raft/virus immunogen. A lipid raft/virus or lipid raft/protein co-cultured immunogen that will generate an antibody response able to neutralize a broad spectrum of primary viral isolates and generate immune responses is created in this fashion. Antibodies to novel epitopes in virus and proteins are also created in this fashion.

The use of lipid raft terminology in this disclosure also includes use of caveolae (i.e., co-cultures of caveolae/virus or co-cultures of caveolae/protein) as immunogens. This method can also be applied to the co-culture of lipid rafts and any pathogen. As such, the pathogen can be an enveloped virus, including but not limited to an immunodeficiency virus such as human immunodeficiency virus, a T lymphocytic virus such as human T lymphocytic virus (HTLV), a herpes virus such as herpes simplex virus (HSV), a measles virus, or an influenza virus. The pathogen also can be a microbial pathogen, for example a bacterium, a yeast such as Candida, a mycoplasma, a protozoan such as Trichomonas, or a Chlamydia.

Lipid raft preparations are easily obtained from a variety of cell sources. Immune cells susceptible to viral infection represent good source for raft preparation. Whole immune cells exposed to virus could also be used as a source of lipid raft/virus immunogen. Co-culture combinations of lipid rafts, viral proteins and various peptides (e.g., the HIV envelope glycoprotein gp120) would also be used as immunogens. Thus, this method is also applicable to generating antibodies to novel conformations of viral, microbial, fungal, and animal proteins when co-cultured and interacting with lipid rafts.

Many proteins translocate into lipid rafts following stimulation. This translocation involves modifications such as palmitylation and/or myristylation. Antibodies raised against such lipid raft/protein immunogens may recognize novel epitopes in the translocated protein. Natural lipid raft/virus co-cultures [define this term/idea/concept better] will serve as immunogens, but lipid raft/virus co-cultures or whole cell/virus co-cultures fixed with low concentrations of fixative such as formalin [glutaraldehyde, or methanol] may also be used. Lipid raft preparations can also be modified to include or exclude selected proteins in order to vary the immunogenic effect of the raft/virus, raft/protein co-culture. In a preferred embodiment, immunizations are performed in mice engineered to be transgenic for human antigens, thus reducing the possibility that the antibodies generated would recognize human proteins.

This novel method of immunogen production may prove useful in the generation of anti-viral vaccines. In the case of human immunodeficiency virus type 1 (HIV-1), success has been gauged by the ability of candidate immunogens to generate measurable immune responses in human volunteers and animal models. The two crucial responses have been the generation of virus-specific CD8⁺ cytotoxic T lymphocytes (CTLs), which attack and destroy infected cells, and production of neutralizing antibodies, which bind to the virus and prevent infection of new cells. For HIV-1, an effective anti-viral vaccine has remained elusive.

A number of studies published in recent years have shown that neutralizing monoclonal antibodies of the IgG class alone can be effective in blocking the infection of non-human primates by mucosal challenge with SHIV. Such studies provided a rationale for testing groups of monoclonal antibodies with synergistic neutralizing antibodies in vitro as immediate postexposure prophylaxis, modeling for perinatal exposure in infants. Cocktails of human IgG1b12, 2G12, 2F5, and 4E10 neutralizing monoclonal antibodies prevented disease in newborn macaques and prevented the establishment of SHIV89.6P infection in half of the animals when given within an hour of exposure (Ferrantelli et al., AIDS; 17: 301-309,2003). Studies in in recently infected HIV patients indicated that neutralizing antibodies are indeed involved in controlling viral replication during the first months after infection, and that the pressure they exert on the virus is significant (Richman et al., Proc Natl Acad Sci USA; 100:4144-4149, 2003).

Budding of nascent virus also occurs from lipid rafts. Thus, in addition to preventing new infection, the present invention is applicable to preventing the spread of infection or re-infection. Clinical application of antibodies created by this method may also prevent outbreaks of virus in infected individuals (e.g., herpes outbreaks).

Beta-cyclodextrins deplete cholesterol and disrupt lipid rafts. A novel use of β-cyclodextrins is extended to applications (e.g., topical cream) to prevent recurrent herpes zoster, herpes oral or genital outbreaks. Topical use of β-cyclodextrins may also reduce the severity of outbreaks as well as shorten their duration. Many pathogens exploit lipid rafts for cell infection as well as cell outbreak. This use of β-cyclodextrins and the disruption of raft structure as a portal for entry or exit are applicable to any pathogen outbreak, which involves lipid rafts. As such, pathogen release from infected cells or neurons may be prevented by the disruption of raft structure by β-cyclodextrins.

The present invention relates to methods of reducing the risk of virus budding or diminishing the severity of outbreak of viral infections. It also may be used to diminish pain and associated symptoms of post-outbreak neuralgia. In one embodiment, a method of the invention is performed by contacting area of viral release (e.g., dermatomes in shingles) with a β-cyclodextrin (β-CD). The afflicted dermatomes may be identified by a tingling sensation (a prodrome), which signals the onset of viral release. Examples of said releasable viruses include but are not limited to: an enveloped virus, for example, an immunodeficiency virus such as human immunodeficiency virus (HIV); a T lymphotrophic virus such as human T lymphotrophic virus (HTLV); a herpes virus such as a herpes simplex virus (HSV); a measles virus; a chicken pox virus or an influenza virus. The β-CD can be any β-CD derivative, for example, 2-hydroxypropyl-β-cyclodextrin. In the case of herpes zoster or any outbreak resulting in an outbreak-induced neuralgia, the pain and associated symptoms may be amenable to topical treatment of β-CD.

The present invention also relates to a pharmaceutical composition, which includes: 1) β-CD, which is in a sufficient amount to block viral release through lipid rafts in the membrane of a nerve cell.

The present invention further relates to a composition, comprising a solid substrate that contains an effective amount of β-CD useful for reducing the risk of viral release and the severity of viral outbreak. The pharmaceutical composition can be formulated in a solution, a gel, a foam, an ointment, a cream, a paste, a spray, or the like.

### DETAILED DESCRIPTION OF INVENTION

To isolate detergent resistant membranes (DRMs) from a cell type including but not limited to primary or transformed lymphocytes. Cells are washed in Buffer A (100 mM NaCl, 10 mM KCl, 10 mM EGTA, 10 mM imidizole, pH 6.8), then in TKM buffer (50 mM Tris-HCl, pH 7.4, 25 mM KCl, 5 mM MgCl, and 1 mM EGTA). To reduce proteolysis, the following protease inhibitors are included in Buffer A: 2 mg/ml of leupeptin (Calbiochem Novabiochem Corp., La Jolla, Calif.); 5 mM Pefa-Bloc (Roche Molecular Biochemicals, Indianapolis, Ind.); 1% aproptinin (Sigma); 1% pepstatin A (Roche Molecular Biochemicals); and 100 nM benzamidine (Sigma). DRMs were prepared using a discontinuous sucrose density gradient. DRMs are located at the interface between 5 and 36% sucrose.

Alternatively, isolation of low-density, Triton X-100-insoluble membrane complexes is easily performed. Briefly, cells were homogenized in 2-morpholinoethanesulfonic acid (MES)-buffered saline containing 1% Triton X-100 (unless otherwise indicated), and sucrose was added to a final concentration of 40%. A 5 to 30% discontinuous sucrose gradient was layered on top of this detergent extract followed by ultracentrifugation [54,000 rpm in a rotor (Beckman Coulter, Fullerton, Calif.)] for 18 to 24 h at 4° C. in a TL-100 ultracentrifuge (Beckman Coulter). Successive gradient fractions were collected from the top and subjected to SDS-PAGE and Western blot analysis.

HIV-1.sub.RF viral supernatant from an infected Jurkat cell line can be collected and clarified through a 0.45.mu.m filter. Virus supernatant (10 ml) can be co-cultured with purified lipid raft fractions as described above. These lipid raft/virus co-cultures serve as immunogens for the creation of novel antibodies. Following hybridoma fusion to create monoclonal expressing immortalized B-cells, antibodies produced in this fashion can be mass screened to determine their effectiveness as neutralizing antibodies. The capacity of purified IgG as well as whole serum, to neutralize HIV can be tested in an assay with phytohemagglutinin-stimulated peripheral blood mononuclear cells. Briefly, antibodies or sera were incubated for 1 h at 37° C. with diluted tissue culture supernatant of virus-infected peripheral blood mononuclear cells (40 to 100 50% tissue culture infective doses, 100 μl). Peripheral blood mononuclear cells (10⁵ in 50 μl) were added to the virus-antibody reaction mixture, and the mixture was incubated overnight. All dilutions were performed with RPMI 1640 medium (GIBCO, Life Technologies Ltd., Paisley, Scotland) supplemented with 10% fetal calf serum, 3 mM glutamine, 20 IU of interleukin-2, and antibiotics. Medium changes were performed on days 1 and 4. Seven days after infection, supernatants were collected and analyzed for HIV antigen by a capture ELISA. The neutralization titer was defined as the reciprocal of the last dilution step that showed an 80% or greater reduction in the OD at 490 nm of the culture supernatant compared to that of HIV antibody-negative serum.

Beta-cyclodextrins (β-CDs) are widely used as solubilizing agents, stabilizers, and inert excipients in pharmaceutical compositions (see U.S. Pat. Nos. 6,194,430; 6,194,395; and 6,191,137, each of which is incorporated herein by reference). Beta-CDs are cyclic compounds containing seven units of α-(1,4) linked D-glucopyranose units, and act as complexing agents that can form inclusion complexes and have concomitant solubilizing properties (see U.S. Pat. No. 6,194,395; see also, Szejtli, J. Cyclodextrin Technol. 1988).

The compositions and methods of the invention are exemplified using 2-hydroxypropyl-β-CD (2-OH-β-CD). However, any β-CD derivative can be used in a composition or method of the invention, provided the β-CD derivative disrupts lipid rafts in the membranes of nerve cells. Beta-CDs act, in part, by removing cholesterol from cell membranes, and different β-CDs are variably effective in such removal. For example, methyl-β-CD removes cholesterol from cell membranes very efficiently and quickly and, as a result, can be toxic to cells, which require cholesterol for membrane integrity and viability. In comparison, a β-CD derivative such as 2-OH—β-CD can effectively remove cholesterol from cells without producing undue toxicity. Thus, it will be recognized that a β-CD useful in a composition or method of the invention is one that removes cholesterol in an amount that disrupts lipid rafts, without substantially reducing cell viability (see, for example, Rothblat and Phillips, J. Biol. Chem. 257:4775-4782 (1982), which is incorporated herein by reference).

Beta-CDs useful in the present invention include, but are not limited to, β-CD derivatives wherein one or more of the hydroxy groups is substituted by an alkyl, hydroxyalkyl, carboxyalkyl, alkylcarbonyl, carboxyalkoxyalkyl, alkylcarbonyloxyalkyl, alkoxycarbonylalkyl or hydroxy-(mono or polyalkoxy)alkyl group or the like; and wherein each alkyl or alkylene moiety contains up to about six carbons. Substituted β-CDs that can be used in the present invention include, for example, polyethers (see, for example, U.S. Pat. No. 3,459,731, which is incorporated herein by reference); ethers, wherein the hydrogen of one or more β-CD hydroxy groups is replaced by C1 to C6 alkyl, hydroxy-C1-C6-alkyl, carboxy-C1-C6 alkyl, C1-C6 alkyloxycarbonyl-C1-C6 alkyl groups, or mixed ethers thereof. In such substituted β-CDs, the hydrogen of one or more β-CD hydroxy group can be replaced by C1-C3 alkyl, hydroxy-C2-C4 alkyl, or carboxy-C1-C2 alkyl, for example, by methyl, ethyl, hydroxyethyl, hydroxypropyl, hydroxybutyl, carboxymethyl or carboxyethyl. It should be recognized that the term “C1-C6 alkyl” includes straight and branched saturated hydrocarbon radicals, having from 1 to 6 carbon atoms. Examples of β-CD ethers include dimethyl-β-CD. Examples of β-CD polyethers include hydroxypropyl-p-β-CD and hydroxyethyl-β-CD (see, for example, Nogradi, “Drugs of the Future” 9(8):577-578, 1984; Chemical and Pharmaceutical Bulletin. 28:1552-1558 (1980); Yakugyo Jiho No. 6452 (Mar. 28, 1983); Angew. Chem. Int. Ed. Engl. 19:344-362 (1980); U.S. Pat. No. 3,459,731; EP-A-0,149,197; EP-A-0,197,571; U.S. Pat. No. 4,535,152; WO-90/12035; GB-2,189,245; Szejtli, “Cyclodextrin Technology” (Kluwer Academic Publ. 1988); Bender et al., “Cyclodextrin Chemistry” (Springer-Verlag, Berlin 1978); French, Adv. Carb. Chem. 12:189-260; Croft and Bartsch, Tetrahedron 39:1417-1474, 1983; Irie et al., Pharm. Res. 5:713-716, 1988; Pitha et al., Internat'l. J. Pharm. 29:73, 1986; U.S. Pat. No. 5,134,127 A; U.S. Pat. Nos. 4,659,696 and 4,383,992, each of which is incorporated herein by reference; see, also, U.S. Pat. No. 6,194,395).

A method of the invention is performed, for example, by contacting an area of skin susceptible to viral release with a β-CD. As used herein, the term “contacting,” when used in reference to a β-CD and the pathogen or cells susceptible to a sexually transmitted pathogen, means that the β-CD is applied to the susceptible area such that it prevents viral budding through lipid rafts at nerve terminals.

As described above, budding of HIV-1 particles occurs at lipid rafts, which are characterized by a distinct lipid composition that includes high concentrations of cholesterol, sphingolipids, and glycolipids. Since cholesterol plays a key role in the entry of some other viruses, the role in HIV-1 entry of cholesterol and lipid rafts in the plasma membrane of susceptible cells was investigated. Example 2 demonstrates that intact lipid rafts are necessary for viral infection. A β-CD derivative, 2-hydroxypropyl-β-cyclodextrin (2-OH-β-CD), was used to deplete cellular cholesterol and disperse lipid rafts. As disclosed herein, removal of cellular cholesterol rendered primary cells and cell lines highly resistant to HIV-1-mediated syncytium formation and to infection by both CXCR4- and CCR5-specific strains of HIV-1 virus. 2-OH-β-CD treatment of the virus or cells partially reduced HIV-1 binding, while rendering chemokine receptors highly sensitive to antibody-mediated internalization, but had no effect on CD4 expression. These effects were readily reversed by incubating cholesterol-depleted cells with low concentrations of cholesterol-loaded 2-OH-β-CD to restore cholesterol levels. Cholesterol depletion also made cells resistant to SDF-1-induced binding to ICAM-1 through LFA-1. This may have contributed to the reduction in HIV-1 binding to cells after treatment with the β-CD, since LFA-1 contributes significantly to cell binding by HIV-1 which, like SDF-1.alpha, can trigger CXCR4 function through gp120. These results indicate that cholesterol is involved in the HIV-1 co-receptor function of chemokine receptors and is required for infection of cells by HIV-1 (Example 2).

As discussed above, cholesterol, sphingolipids, and GPI-anchored proteins are enriched in lipid rafts (see Simons and Ikonen, Nature. 387:569-572, 1997). The high concentration of cholesterol and sphingolipids in lipid rafts results in a tightly packed, ordered lipid domain that is resistant to non-ionic detergents at low temperature. The structural protein caveolin causes formation of flask-shaped invaginations (caveolae) in the cell membrane with a lipid composition very similar to that of lipid rafts (Schnitzer et al., Science 269:1435-1439, 1995). Signaling molecules, including Lck, LAT, NOS, and G protein a subunit, are localized to rafts on the intracellular side of the membrane, and are targeted by lipid modifications such as palmitylation, myristylation, or both. In comparison, many other transmembrane proteins do not show a preference for lipid rafts; for example, CD45 and E cadherin are excluded from these areas. Certain lipid modified transmembrane proteins such as the HA molecule of influenza virus localize to lipid rafts.

As disclosed herein, HIV-1 buds selectively from lipid rafts of infected T cells (Example 1). In addition, Semliki Forest Virus (SFV), measles viruses, influenza viruses, and polioviruses all assemble by raft association and, in the case of influenza virus, bud from lipid rafts (see, for example, Marquardt et al., J. Cell Biol. 123:57-65, 1993; Manie et al., J. Virol. 74:305-311, 2000; Zhang et al., J. Virol. 74:4634-4644, 2000, each of which is incorporated herein by reference). The involvement of lipid rafts in HIV-1 biology beyond its role in virus budding has been further examined. As further disclosed herein, partial depletion of cholesterol from cell membranes using a β-CD inhibited HIV-1-induced syncytium formation in cell lines and primary T cells (Example 2). β-CD treatment of cells also increased CR internalization induced by monoclonal antibody (MAb) binding. Primary cells and cell lines were rendered resistant to infection CXCR4-specific and CCR5-specific HIV-1 strains by treatment with 2-OH-β-CD (Example 2). The effects observed were not due to loss of cell viability after treatment with the β-CD, and demonstrate that intact lipid rafts and cholesterol are required for HIV-1 infection and syncytium formation.

The present invention also provides compositions useful for reducing the risk of transmission of sexually transmitted disease. A composition of the invention contains a β-CD, which can be in a form suitable for topical administration to a subject, particularly intravaginal or intrarectal use, including a suppository or a bioadhesive polymer, which can provide timed release of the β-CD (see, for example, U.S. Pat. Nos. 5,958,461 and 5,667,492, each of which is incorporated herein by reference); or can be formulated in combination with a solid substrate to produce a condom, diaphragm, sponge, tampon, a glove or the like (see, for example, U.S. Pat. Nos. 6,182,661 and 6,175,962, each of which is incorporated herein by reference), which can be composed, for example, of an organic polymer such as polyvinyl chloride, latex, polyurethane, polyacrylate, polyester, polyethylene terephthalate, polymethacrylate, silicone rubber, a silicon elastomer, polystyrene, polycarbonate, a polysulfone, or the like (see, for example, U.S. Pat. No. 6,183,764, which is incorporated herein by reference).

For topical administration, the β-CD can be formulated in any pharmaceutically acceptable carrier, provided that the carrier does not affect the activity of the β-CD in an undesirable manner. Thus, the composition can be, for example, in the form of a cream, a foam, a jelly, a lotion, an ointment, a solution, a spray, or a gel (see U.S. Pat. No. 5,958,461, which is incorporated herein by reference). In addition, the composition can contain one or more additional agents, for example, an antimicrobial agent such as an antibiotic or an antimicrobial dye such as methylene blue or gentian violet (U.S. Pat. No. 6,183,764); an antiviral agent such as a nucleoside analog (e.g., azacytidine), a zinc salt (see U.S. Pat. No. 5,980,477, which is incorporated herein by reference), or a cellulose phthalate such as cellulose acetate phthalate or a hydroxypropyl methylcellulose phthalate (see U.S. Pat. No. 5,985,313, which is incorporated herein by reference); a contraceptive (see U.S. Pat. No. 5,778,886, which is incorporated herein by reference); a lubricant, or any agent generally useful to a sexually active individual, provided the additional agent, either alone or in combination, does not affect the activity of the β-CD or, if it affects the activity of the β-CD, does so in a predictable way such that an amount of β-CD that is effective for reducing viral outbreak can be determined.

A pharmaceutically acceptable carrier useful in a composition of the invention can be aqueous or non-aqueous, for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the β-CD, for example, carbohydrates, such as glucose, sucrose or dextrans, antioxidants, such as ascorbic acid or glutathione, chelating agents, low molecular weight proteins or other stabilizers or excipients.

The pharmaceutical composition also can comprise an admixture with an organic or inorganic carrier or excipient suitable for intravaginal or intrarectal administration, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

The β-CD also can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton, Fla. 1984); Fraley, et al., Trends Biochem. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see U.S. Pat. Nos. 5,882,679; 5,395,619; and 5,225,212, each of which is incorporated herein by reference) are an example of such encapsulating materials particularly useful for preparing a pharmaceutical composition of the invention, and other “masked” liposomes similarly can be used, such liposomes extending the time that the β-CD remains at the site of administration.

The amount a β-CD in a composition can be varied, depending on the type of composition, such that the amount present is sufficient to reduce viral outbreak or reduce severity of outbreak. An example of such an amount is about 1 to 100 mM, generally about 5 to 30 mM, when administered in an ointment, gel, foam, spray or the like, our about 0.1 to 2 grams, generally about 0.25 to 0.75 grams, when administered as a suppository or in combination with a solid substrate. An effective amount of a β-CD also can be measured in a weight:weight (w:w) or weight:volume (w:v) amount, for example, about 0.1% to 3% w:w with respect to a solid substrate or about 0.1% to 3% w:v with respect to a pharmaceutically acceptable carrier. In addition, an amount of a β-CD sufficient to reduce viral outbreak or decrease outbreak severity can be determined using routine clinical methods, including Phase I, II and III clinical trials.

Currently, several HIV-1 vaccine approaches are being developed, each with its own relative strengths and weaknesses. These approaches include the development of live attenuated vaccines, inactivated viruses with adjuvant peptides and subunit vaccines, live vector-based vaccines, and DNA vaccines. Envelope glycoproteins were considered as the prime antigen in the vaccine regimen due to their surface-exposure, until it became evident that they are not ideal immunogens. This is an expected consequence of the immunological selective forces that drive the evolution of these viruses: it appears that the same features of envelope glycoproteins that dictate poor immunogenicity in natural infections have hampered vaccine development. However, modification of the vaccine recipe through the use of raft/virus co-cultures to expose novel viral epitopes may overcome these problems.

Accordingly, there is a need in the art for new effective methods of identifying candidate sequences for vaccine development to prevent and treat HIV infection. The present invention fulfills this and other needs.

“Antibody” refers to a polypeptide substantially encoded by an immunoglobulin gene or immunoglobulin genes, or fragments thereof, that specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain has a variable region of about 100 to 110 or more amino acids primarily responsible for antigen recognition. The terms variable light chain (VL) and variable heavy chain (VH) refer to these light and heavy chains, respectively.

Antibodies exist, for example, as intact immunoglobulins or as a number of well characterized antigen-binding fragments produced by digestion with various peptidases. For example, pepsin digests an antibody below the disulfide linkages in the hinge region to produce an F(ab′).sub.2 fragment, a dimer of Fab which itself is a light chain joined to VH-CH1 by a disulfide bond. The F(ab′).sub.2 fragment can be reduced under mild conditions to break the disulfide linkage in the hinge region, thereby converting the F(ab′).sub.2 dimer into an Fab′ monomer. The Fab′ monomer is essentially an Fab with part of the hinge region (see, Fundamental Immunology, Third Edition, W. E. Paul (ed.), Raven Press, N.Y. (1993)). While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments, such as a single chain antibody, an antigen binding F(ab′).sub.2 fragment, an antigen binding Fab′ fragment, an antigen binding Fab fragment, an antigen binding Fv fragment, a single heavy chain or a chimeric antibody. Such antibodies can be produced by the modification of whole antibodies or synthesized de novo using recombinant DNA methodologies.

Thus, an immunogenic composition to this subtype B ancestor protein will elicit broad neutralizing antibody against HIV-1 isolates of the same subtype. An immunogenic composition to this subtype B ancestor protein will also elicit a broad cellular response mediated by antigen-specific T-cells.

Monoclonal antibodies (Mabs) have been available for over 25 years and have revolutionalized biomedical research, especially in the areas of disease diagnosis and the treatment of infection and diseases.

The conventional method for the production of monoclonal antibodies involves hybridomas (Kohler & Milstein, Nature 256:495-7, 1975). In this method, splenic or lymphocyte cells from a mammal which has been injected with antigen are fused with a tumor cell line, thus producing hybrid cells. These hybrid cells, or “hybridomas”, are both immortal and capable of producing the genetically coded antibody of a B cell. To select a hybridoma producing a single antibody, the hybridomas made by cell fusion are segregated by selection, dilution, and regrowth until a single genetically pure antibody-expressing cell line is selected. Because hybridomas produce homogeneous antibodies against a desired antigen, they are called “monoclonal” antibodies. Hybridoma technology has primarily been focused on the fusion of murine lines, but also human-human hybridomas, human-murine hybridomas, rabbit-rabbit hybridomas and other xenogenic hybrid combinations have been made.

### EXAMPLES

### Example 1

HIV-1 Selectively Buds from Lipid Rafts

This example demonstrates that HIV-1 budding occurs through lipid rafts, thereby accounting for the cholesterol-rich, sphingolipid-rich virus membrane, which bears GPI-linked proteins such as Thy-1 and CD59, but lacks CD45.

The relative incorporation of GM1, a ganglioside marker specific for lipid rafts, also was examined. Using a soluble CTB binding assay, as much as 75% of HIV-1 was precipitated using goat anti-CTB and SaC after treating the virus with GM 1-specific CTB). The CTB binding to virus was specific and dose dependent, and no virus was precipitated in the absence of CTB as measured by p24 ELISA. These results demonstrate that the majority of HIV-1 particles incorporate the lipid raft-specific marker GM1.

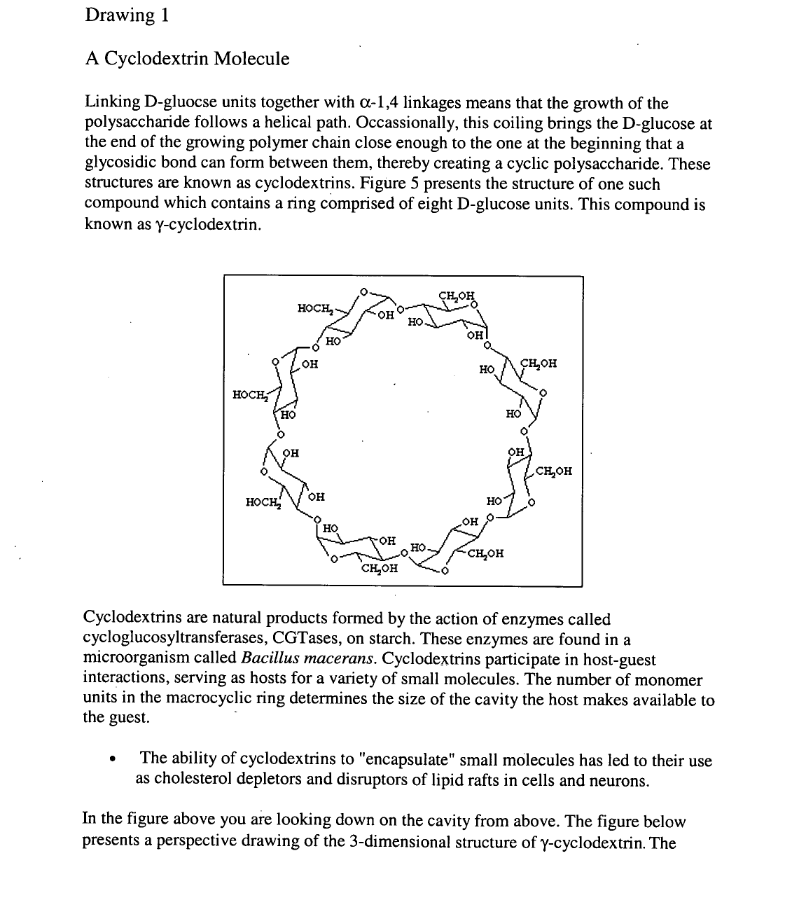

Thy-1, CD59, and GM1 colocalized with Hw-1 proteins on infected cell uropods, which excluded CD45. To determine the distribution of HIV-I proteins relative to GPI-linked proteins that serve as lipid raft markers, infected cells were subjected to immunofluorescence staining followed by confocal microscopy. Expression of HIV-1 proteins was localized to uropods projecting from one end of the cell. This capping pattern was seen on most cells in the infected cell culture. Uropods protruding from HIV-1-infected cells have been described for adherent T cells. Thy-1 and CD59 both colocalized with cell surface HIV-1 proteins, as shown by a superimposed green (Thy-1 or CD59) and red (HIV-1 proteins) fluorescence (see Nguyen and Hildreth, supra, 2000; FIG. 4). Cells that were prefixed with 2% paraformaldehyde before staining showed a similar appearance, indicating that the colocalization was not due to antibody crosslinking of viral and GPI-linked proteins. Since the cells were not permeabilized before staining, the HIV proteins seen in these studies are likely gp41 and gp120. This was confirmed in studies with anti-gp41 MAb T32 in the colocalization studies. Uninfected cells showed no capping of Thy-1 or CD59. CD45 did not localize to areas of HIV-1 protein expression and was excluded from uropods. The distribution of CD45 was unaffected by HIV-1 infection, and the molecule remained evenly dispersed in patches all over the cell surface. These results confirm those obtained using the virus phenotyping studies. The ability of GM1 to colocalize on the cell surface with HIV-1 proteins was examined to confirm the finding that GM1 was present on virions. GM1 staining was relatively faint with rabbit anti-GM1 antibody, but confocal microscopy showed colocalization of this molecule with HIV-1 labeled cells.

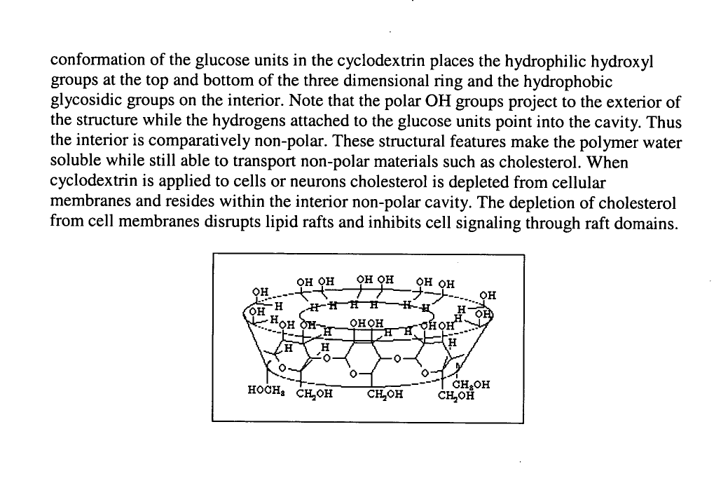

HIV-1 proteins were detected in isolated lipid raft fractions. Lipid rafts were purified by cell lysis and equilibrium centrifugation in order to confirm the presence of HIV-1 proteins in these membrane structures. The fractions were assayed for the presence of viral and host proteins by immunoblot analysis. The separation of detergent-resistant lipid rafts was confirmed by the abundance of Thy-1 and CD59 in fractions 3 through 5, while CD45 was present only in the bottom fractions 9 and 10 (see Nguyen and Hildreth, supra, 2000; FIG. 6). Immunoblot detection of membrane fractions revealed that the HIV MA protein, p17, and gp41 were both present in the detergent-insoluble lipid rafts of infected cells.

### Example 2

Host Membrane Cholesterol is Required for HIV-1 Infection

By removing cholesterol, 2-OH-.beta.CD is believed to partially perturb organized lipid rafts, resulting in dispersal of their components (Ilangumaran and Hoessli, Biochem. J. 335:433-440, 1998). The capture of HIV-1 by MAbs against CD59 and gp41 decreased substantially after treating cells with 2-OH-.beta.CD, as measured by the percentage of total p24. CD45 capture remained unaffected. The effects on virus precipitation through gp41 indicate that intact lipid rafts are required for efficient gp41 incorporation into virions, since the overall cellular release of p24 actually increased after 2-OH-.beta.CD treatment.

Results 2-OH-betaCD treatment blocked syncytium formation of primary cells and cell lines. The role of lipid rafts in the HIV-1 fusion process was examined by treating CD4+HIV-susceptible target cells with 2-OH-betaCD to deplete membrane cholesterol and disperse lipid rafts. Treatment of cells with 10 to 20 mM 2-OH-betaCD for 1 hr at 37 degree ° C., followed by washing to remove free 2-OH-.beta.CD, depleted greater than 70% of total cellular cholesterol without any loss in cell viability as measured by Trypan Blue exclusion. Furthermore, treated cells continued to grow normally after 2-OH-betaCD treatment when placed back into culture in cholesterol-containing medium. The non-toxicity of betaCD treatment was further demonstrated by finding 2-OH-.beta.CD treated Jurkat cells still showed Ca²⁺ flux responses to anti-CD3 MAb.

1. A method of reducing the outbreak or release of virus from cells of a living mammal, the method comprising application to the body of said mammal with a β-cyclodextrin.

2. The method of claim 1, wherein the mammal is a human.

3. The method of claim 1, wherein the β-cyclodextrin is selected from the group consisting of: β-cyclodextrins wherein one or more of the hydroxy groups is substituted by an alkyl, hydroxyalkyl, carboxyalkyl, alkylcarbonyl, carboxyalkoxyalkyl, alkylcarbonyloxyalkyl, alkoxycarbonylalkyl or hydroxy-(mono or polyalkoxy)alkyl group. Make sure complete for all dextrins

4. The method of claim 1, wherein the β-cyclodextrin is 2-hydroxypropyl-β-cyclodextrin. Name a few more specifically

5. The method of claim 1, wherein the β-cyclodextrin is formulated in a solution, a gel, a foam, an ointment, a cream, a paste, a spray, a suppository, or a film.

6. The method as in one of claims 1-5, wherein the virus is an enveloped virus.

7. The method as in one of claims 1-5, wherein the enveloped virus is an immunodeficiency virus, a T lymphotrophic virus, a herpesvirus, a measles virus, a chicken pox virus or an influenza virus.

8. The method as in one of claims 1-5, wherein the immunodeficiency virus is a human immunodeficiency virus (HIV).

9. The method as in one of claims 1-5, wherein the HIV is HIV type I.

10. The method of claim 7, wherein said application comprises topical application to epidermal, mucosal, urogenital, urethral, vaginal, anorectal, or colonic areas of said mammal.

11. An anti-viral composition suitable for preventing viral budding from cells of a living mammal comprising: an effective amount of a β-cyclodextrin.

12. The antiviral composition of claim 11 wherein said β-cyclodextrin is selected from the group consisting of: β-cyclodextrins wherein one or more of the hydroxy groups is substituted by an alkyl, hydroxyalkyl, carboxyalkyl, alkylcarbonyl, carboxyalkoxyalkyl, alkylcarbonyloxyalkyl, alkoxycarbonylalkyl or hydroxy-(mono or polyalkoxy)alkyl group.

13. The antiviral composition of claim 11 wherein said β-cyclodextrin is 2-hydroxypropyl-β-cyclodextrin.

14. The antiviral composition of claims 11, 12, or 13, wherein the β-cyclodextrin is formulated in a solution, a gel, a foam, an ointment, a cream, a paste, a spray, a suppository, or a film.

15. A method of reducing the severity of outbreak and/or reducing the infectivity to a living mammal or the cells of said mammal, of a pathogen selected from the group consisting of bacterial pathogens, fungal pathogens, other microbial pathogens, the method comprising application to the body of said mammal a β-cyclodextrin.

16. A method of creating novel epitopes for use as immunogens, the method comprising a co-culture of lipid rafts and virus to expose novel viral and epitopes due to lipid raft/virus interactions.

17. The method of claims 16, wherein the virus is an enveloped virus.

18. The method of claim 16, wherein the enveloped virus is an immunodeficiency virus, a T lymphotrophic virus, a herpesvirus, a measles virus, a chicken pox virus or an influenza virus.

19. The method of claim 16, wherein producing monoclonal antibodies from said immunogens comprises generating hybridoma cells from antibody producing cells, harvesting monoclonal antibodies from said produced hybridoma cells, and screening said population of monoclonal antibodies to identify monoclonal antibodies of interest.

20. The method of claim 16 where the co-culture is between lipid rafts and a protein to expose novel epitopes in the protein due to the lipid raft/protein interaction.

21. A immunogenic composition suitable for use as an antigenic agent for the creation of monoclonal or polyclonal antibodies where the composition is the mixture of lipid rafts and virus.

22. The immunogenic composition of claim 21, wherein the virus is an enveloped virus.

23. The immunogenic composition of claim 21, wherein the enveloped virus is an immunodeficiency virus, a T lymphotrophic virus, a herpesvirus, a measles virus, a chicken pox virus or an influenza virus.

24. The immunogenic composition in claim 21 where the composition is the mixture of lipid rafts and a protein.
